(19) United States
(12) Patent Application Publication
(10) Pub. No.: US 2009/0005834 A1
(43) Pub. Date: Jan. 1, 2009

(54) Deep vein thrombosis prevention

(76) Inventors: Eitan Weintraub, Yavne (IL); David Weintraub, Yavne (IL); Yoram Eshel, Tel Aviv (IL)

Correspondence Address: EMPK & Shiloh, LLP;c/o Landon IP, Inc., 1700 Diagonal Road, Suite 450, Alexandria, VA, 22314, US

(21) Appl. No.: 11/819,541

(22) Filed: Jun. 28, 2007

(51) Int. Cl. A61N 1/00; A61B 18/18; A61H 23/00

(52) U.S. Cl. 607/48; 601/48; 607/62; 607/88

(57) ABSTRACT

A device adapted for mitigating the formation of a blood clot, the device comprising: a sensor adapted to identify muscle movement associated with blood circulation in a vein and to produce a signal indicating the movement; and a controller adapted to monitor the sensor signal and to activate an actuator upon said controller determining that less than a defined number of movements have been sensed over a defined period of time, wherein the actuator is adapted to produce a stimulating action of a given set of parameters urging the user to move his/her feet if, for example, the predefined number of dorsiflexions is not met.

### FIELD

The invention relates to deep vein thrombosis prevention.

### BACKGROUND

Deep vein thrombosis (DVT) is a condition in which a blood clot, or several blood clots, form inside a vein located deep within a muscle, typically in the lower part of the leg, the calf, though also possibly in other parts of the body such as, for example, an arm, a pelvis, or a thigh. Usually, the clots are relatively small and do not cause any damage, eventually breaking down and disappearing. DVT may occasionally be followed by complications, an example of which is when the clots break loose and travel through the bloodstream to the lungs and into the pulmonary arteries. Once in the pulmonary arteries the clots may create a blockage of the right ventricle, a relatively rare event, which is usually a fatal clinical condition, or may spread in the pulmonary smaller vessels, a rather more frequent event. This condition is generally referred to as “pulmonary embolism”. If not treated immediately, pulmonary embolism in many instances may have fatal consequences. Another complication may result from damage to the valves in the affected veins and consequently poor blood flow in the lower part of the leg. This condition, known as “post-thrombotic syndrome” is generally characterized by symptoms such as swelling, chronic pain and ulcers in the leg. These symptoms are usually associated as being long term symptoms of DVT.

Medical studies attribute approximately half of the DVT cases to inherited tendency and the approximate remaining half to such factors as: surgery (despite the use of anticoagulants prior to and after an operation); extended periods of inactivity such as, for example, prolonged bed rest, long trips by plane or by road and long working hours by a computer; increased level of clotting factors in the blood caused by, for example, some types of cancers, particularly pancreatic, ovarian and lung cancer, smoking, overweight, pregnancy, or due to intake of the female hormone estrogen; cardiovascular related diseases associated with clot formation such as, for example, myocardial infarction or stroke; and, injury to the veins in the legs, for example, as may occur from leg or pelvic fracture, or from surgical procedures involving knee or hip replacement. Furthermore, any combination of these factors greatly increases the risk of a person developing DVT, referred to hereinafter as a “high risk” person or people. For example studies show that high risk people who are immobilized after surgery, heart attacks, or serious injuries are more prone to develop DVT and pulmonary embolism than those who are allowed to walk around. Additionally, incidents of DVT and pulmonary embolism have seen an increase in high risk airplane passengers due to lack of mobility and/or exercising while traveling on long plane trips. More information on DVT and pulmonary embolism may be found in the following website articles, “Pulmonary Embolism—MayoClinic.com” at www.mayoclinic.com/health/pulmonary-embolism/DS00429, and in “Deep vein thrombosis—DVT blood clot cause, symptoms and treatment” at hcd2.bupa.co.uk/fact_sheets/mosby_factsheets/Deep_Vein_Thrombosis.html, both articles incorporated herein by reference.

Numerous devices known in the art are used to stimulate mobility and/or exercising in people subject to extended periods of inactivity. Some of these devices are described below.

One such device is described in Pub. No.: US 2006/0255955 A1, “Activity Monitoring Device”, which comprises a device for monitoring the activity of a user to prevent deep vein thrombosis when traveling on transportation vehicles. The device comprises a motion sensor adapted to detect a user performing a predefined motion and a processor adapted to filter the motion detected to remove background motion not attributable to a desired exercise. The need to perform differentiation between motion attributable to the user performing the defined exercise and the overall pattern of motion detected, contributes to an increase in design complexity and device cost.

Another device is described in Pub. No.: US 2005/0228317 A1, “Warning Device for Prevention of Deep Vein Thrombosis”, which comprises a device for monitoring a length of time a person has been immobile. The device comprises a pressure sensor configured to detect if a person has been immobile for a prolonged period of time. With this device if a person is constrained within a certain space and cannot get up for a predetermined period of time the alarm in the device may go off repeatedly.

Other devices are adapted to massage part or the whole of the leg or to electrically stimulate muscles in the leg. However, these devices are uncomfortable to use and expensive to manufacture. U.S. Pat. No. 6,290,662 B1, “Portable Self-Contained Apparatus for Deep Vein Thrombosis (DVT) Prophylaxis” describes an apparatus which comprises a bladder that expands and directs compressive forces against a body part. U.S. Pat. No. 6,282,448 B1, “Self Applied and Self Adjusting Device and Method for Prevention of Deep Vein Thrombosis with Movement Detection” describes a device which is adapted to provide an electrical signal causing muscle contraction detected by a motion detector comprised by the device.

As discussed above numerous devices are known in the art of which several have been mentioned. Many are not suitable for use in cramped spaces typical of vehicles used in long journeys such as may be, for example, a car, train, bus or airplane. Others are better suited for use in travel but are relatively expensive to manufacture. Therefore, there is a need for a device that is adapted to reduce the risk of development of DVT in a user, which is suitable for use in cramped spaces where mobility is substantially restricted, and which is relatively inexpensive to manufacture.

### SUMMARY

An aspect of some embodiments of the invention relates to providing a device adapted to monitor a user's action of moving a foot such that the movement enhances blood flow in a vein. Such movement may include flexing a foot upward, which action is generally referred to as “dorsiflexion”. According to an aspect of some embodiments of the invention, the device is also adapted to alert the user if the number of movements that enhances blood flow in a vein, such as dorsiflexions, performed over a defined period of time is below a defined number of movements. The defined period of time is hereinafter referred to as a “defined period” and the “defined number of movements” is hereinafter referred to as “defined value”.

According to an aspect of some embodiments of the invention dorsiflexion repeatedly performed over a period of time reduces the risk of, and may even prevent, deep vein thrombosis (DVT). In an embodiment of the invention a device comprising a sensor detects contractions in the Anterior Tibial muscle in the leg and activates an actuator if the number of contractions detected during the defined period is below a defined value. The actuator is adapted to produce a stimulating action so as to alert the user of a possible risk for DVT condition. Optionally, in some embodiments of the invention a device comprising a sensor detects movement in the Achilles tendon and/or Anterior Tibial muscle tendon, both in the leg, and activates an actuator if the number of movements detected during the defined period is below a defined value. Additionally or alternatively, in other embodiments of the invention a device comprising a sensor detects dorsiflexion through sensor contact with the dorsal side of the foot and activates an actuator if the number of dorsiflexions detected during the defined period is below a defined value. Optionally, in some embodiments of the invention a device comprising a sensor is adapted to be used with an insole in a shoe and detects movement of the foot associated with dorsiflexion. Additionally or alternatively, in some other embodiments of the invention a device comprising a sensor is adapted to a shoe and detects movement of the shoe. Optionally, in accordance with some embodiments of the invention a device comprising a sensor is adapted to a sock and detects foot movement associated with dorsiflexion.

There is therefore provided, in accordance with an embodiment of the invention, a device adapted for mitigating the formation of a blood clot, the device comprising: a sensor adapted to identify muscle movement associated with blood circulation in a vein and to produce a signal indicating the movement; and a controller adapted to monitor the sensor signal and to activate an actuator upon said controller determining that less than a defined number of movements have been sensed over a defined period of time, wherein the actuator is adapted to produce a stimulating action of a given set of parameters.

In accordance with some embodiments of the invention the muscle movement comprises dorsiflexion. Furthermore, the number of muscle movements, the period of time or both may be dynamically re-definable. Additionally or alternatively, the number of muscle movements, the period of time or both may be defined according to the medical condition of the user.

In some embodiments of the invention the controller may be adapted to reset a counter upon the controller determining that a defined number of movements have been sensed over a defined period of time. The controller may additionally be adapted to stop the activation of the actuator upon determining that at least defined numbers of movements have been sensed over a defined period of time.

In accordance with some embodiments of the invention the stimulating action may comprise motion, vibration, rotation, alarm, electric pulse production or any combination thereof. The given set of parameters of the stimulating action may comprise frequency, intensity, time or any combination thereof, of which any one of the parameters may be variable. Optionally, the controller may be adapted to vary at least one parameter after a certain period of time has passed from the initiation of the stimulating action, such as the alarm.

According to some embodiments of the invention the actuator is further adapted to be affixed near the sensed muscle and to stimulate a user when actuated. The actuator may comprise a mechanical actuator, an electric actuator, an aural actuator, visual actuator or any combination thereof. Additionally, the electric actuator may be adapted to stimulate and trigger the muscle of a user.

In accordance with some embodiments of the invention the sensor may comprise a mechanical sensing unit, a piezo-electric sensing unit, a strain gage sensing unit, an Infrared (IR) sensing unit, an acceleration sensing unit or any combination thereof, and may be located in or in proximity to the dorsal side of the foot. Alternatively, the sensor may be adapted to sense the muscle through sensing motion of a tendon. Optionally, the sensor may be adapted to sense the muscle through sensing a contraction or stretching of the muscle.

According to some embodiments of the invention the device comprises a transmitter adapted to trigger an action of another device for stimulating blood flow in a foot. The transmitter may be a wireless transmitter and may comprise a radio frequency (RF) transmitter.

In some embodiments of the invention a fastener is adapted to attach the device to a users limb. Alternatively, the device may be comprised in a shoe and/or a sock and/or an insole which may be worn by a user.

There is provided in accordance with an embodiment of the invention a method of mitigating the formation of a blood clot, the method comprising: sensing muscle movements associated with blood circulation in a vein; and actuating a stimulating action of a given set of parameters upon determining that less than a defined number of movements have been sensed over a defined period of time.

In some embodiments of the invention the methods provides for muscle movement comprising dorsiflexion. Optionally, the method provides for the defined number of muscle movements, the period of time or both to be dynamically re-definable. Additionally or alternatively, the number of muscle movements, the period of time or both may be defined according to medical and/or environmental conditions related to the user. Optionally, the defined number of muscle movements is at least one and the period of time is above 1 minute.

In accordance with some embodiments of the invention there is provided a method comprising resetting a counter upon determining that a defined number of movements have been sensed over a period of time. Optionally, the method may comprise stopping the actuation upon determining that at least defined numbers of movements have been sensed over a period of time.

In accordance with some embodiments of the invention there is provided a method wherein the stimulating action may comprise vibration production, sound production, light production, electric pulse production or any combination thereof. The given set of parameters may comprise frequency, intensity, time or any combination thereof, of which any one of the parameters may be variable. Optionally, the method may comprise varying at least one parameter after a certain period of time has passed from the initiation of stimulating action, such as the alarm.

In accordance with some embodiments of the invention the method further comprises stimulating blood flow in a foot.

According to some embodiments of the invention the method provides for a system adapted to increase blood flow comprising the device.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 schematically shows a functional block diagram of a dorsiflexion monitoring device (DMD) in accordance with an embodiment of the invention;

FIGS. 2a and 2b schematically show a flow chart, divided in two portions, illustrating the method of operation of the dorsiflexion monitoring device in accordance with an embodiment of the invention;

FIG. 3 schematically shows a dorsiflexion monitoring device in an exemplary attachment configuration for sensing Anterior Tibial muscle contraction in the leg, in accordance with an embodiment of the invention;

FIG. 4 schematically shows a dorsiflexion monitoring device in an exemplary attachment configuration for sensing Achilles tendon and/or Anterior Tibial muscle tendon movement in the leg, in accordance with another embodiment of the invention; and

FIG. 5 schematically shows a dorsiflexion monitoring device in an exemplary attachment configuration for sensing dorsiflexion directly from the dorsal side of the foot in accordance with other embodiments of the invention;

FIG. 6 schematically shows a dorsiflexion monitoring device comprised in an exemplary insole of a shoe for sensing dorsiflexion, in accordance with some embodiments of the invention; and,

FIG. 7 schematically shows a DMD 700 including an RF transmitter, comprised in an exemplary insole 710 of a shoe for sensing dorsiflexion, in accordance with some embodiments of the invention.

### DETAILED DESCRIPTION

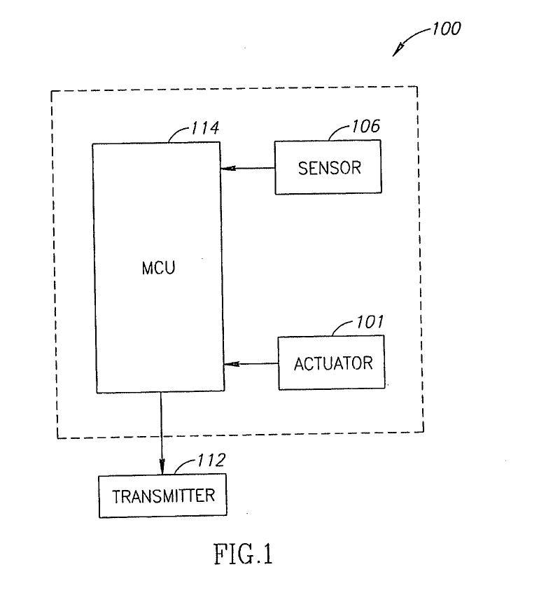

Reference is made to FIG. 1 which schematically shows a functional block diagram of a dorsiflexion monitoring device (DMD) 100 in accordance with an embodiment of the invention. DMD 100 is adapted to mitigate the formation of a blood clot by monitoring dorsiflexion in a user with the aim of reducing the risk of development of DVT due to extended periods of time sitting and/or lying down. Certain muscles and tendons in the legs, during dorsiflexion, experience movements which may include stretching and contractions, usually contributing to improved blood circulation. The DMD monitors the number of muscle and/or tendon movements, hereinafter referred to as “muscle movements” or “dorsiflexions” over a defined period of time (defined period) and if the number of movements is less than a “defined number of movements” (defined value), an actuator is activated to alert the user of the risk of DVT. Generally, the defined period may range from 10 minutes to 4 hours, for example 10-30 minutes, 31-60 minutes, 61-100 minutes, 101-150 minutes, 151 minutes-190 minutes, 191 minutes-240 minutes, and the defined value may range from 1-30 movements, for example, 1-10 movements, 11-20 movements, 21-30 movements. The user, in response to a stimulus from the actuator, will typically increase the muscle movements to meet or exceed the defined value by exercising in place or by getting up and walking. DMD 100 may be attached at different locations in the leg at the user's convenience and comfort. For example, in some embodiments of the invention the DMD may be attached at a location where it senses Anterior Tibial muscle contraction, while in other embodiments DMD may preferably be attached at a location where it senses Achilles tendon and/or Anterior Tibial muscle tendon movement, while still in other embodiments of the invention the DMD may be attached where dorsiflexion is preferably sensed directly from the dorsal side of the foot. Optionally, in some embodiments of the invention, the DMD is comprised in an insole of a shoe. Additionally or alternatively, in some embodiments of the invention the DMD is comprised in a shoe or a sock.

DMD 100 comprises a sensor unit 106, a main control unit (MCU) 114, an actuator unit 101, and optionally a transmitter unit 112. Also comprised in DMD 100 are a protective case (not shown) and an attachment band (not shown).

MCU 114 comprises a controller which includes appropriate control circuitry adapted to receive a sensor signal from sensor unit 106 and to activate actuator unit 101 in response to infrequent muscle movement. Furthermore the MCU comprises one or more counters and/or timers adapted to count muscle movements and to count time. The counter and/or timer may be implemented in MCU 114 by means of hardware, software, or a combination of both. MCU 114 is preferably located within DMD 100 although in some embodiments of the invention the MCU may be a unit externally located to the DMD, and may be physically comprised in a data processing device such as, for example, a laptop computer or a desktop computer. The MCU may also be adapted to run self-tests to determine proper DMD operation. In addition, the device may optionally be programmable, for example, such that the defined period and the defined value are dynamically redefinable, for example according to the user's age, gender, medical condition, medical history, weight, height, barometric pressure, hydration status or any other parameter or any combination of parameters. In some embodiments of the invention, buttons or any other input means (such as a touch screen or voice activation) may also be used to reset the MCU, for example, in counter and/or timer related operations, and/or may also be used to activate and deactivate the DMD. In other embodiments of the invention, the MCU may comprise a display for displaying information such as, for example, defined value, muscle movements, number of times DMD has been reset, operating power status, defined period, and self-test results.

Sensor unit 106 is adapted to convert movement into a sensor signal, which may be electrical signals or data, which are sent to the MCU. Sensor unit 106 may comprise a piezo electric sensor and/or a strain gauge sensor and/or a mechanical sensor and/or an infrared (IR) motion sensor. In some embodiments of the invention sensor unit 106 may comprise a mechanical sensor wherein a mechanical button or switch is toggled or optionally depressed.

In response to a low number of muscle movements MCU 114 sends signals to actuator unit 101, which may include an alarm, which is adapted to produce a stimulating action, or a combination thereof, to alert the user of a possible DVT condition. The stimulating actions may comprise motion such as, for example, vibration or rotation which may be felt by the user. Other stimulating actions may comprise aural characteristics such as, for example, sounding an alarm which may be heard by the user. The alarm may be monotonic or multitonal, and may optionally be single pitch or variable pitch. Even other stimulating actions may comprise generating electrical signals for activating light emitting diodes (LEDs) and/or other light emitting elements, which may serve to attract the user's attention. The light, for example, may be of the same intensity or of variable intensity, and/or of same color or different colors, and/or of constant illumination or flashing. Furthermore, the electrical signal may comprise a pulsing signal adapted to provide a harmless electrical energy shock to the user in order to attract the user's attention. Additionally or alternatively, the actuator unit may be connected to a computer screen for visual and/or aural monitoring of the alarm.

Transmitter unit 112 may be a wireless, transmitter, for example, a radio frequency (RF) transmitter, adapted to transmit to a receiver located externally to the DMD information related to the operation of DMD 100. The information may include, for example, defined value, muscle movements, number of times DMD has been reset, operating power status, defined period, and self-test results. Optionally, in some embodiments of the invention transmitter unit 112 may be adapted to trigger by means of RF signaling, devices which may stimulate blood flow in the user's leg. Additionally or alternatively, in some embodiments of the invention the transmitter unit may be adapted to transmit a signal which will activate an alarm in a receiver, for example, a watch adapted to receive RF transmissions, a mobile phone, or any other type of device which may be adapted to receive an RF transmission and activate an alarm.

DMD 100 comprises a power unit (not shown) which may include a dc voltage source such as non-rechargeable battery/batteries although in some embodiments of the invention the dc voltage source may be rechargeable battery/batteries. Furthermore, in other embodiments of the invention power unit may comprise an ac/dc voltage source for connection of the DMD to an electrical ac outlet such as is found, for example, in the home, workplace or in medical facilities such as hospitals and clinics. Optionally, the power unit may be connected through a USB interface for dc power supply from a PC, laptop computer, or other USB interface dc power supply source.

The protective case is adapted to house the power unit, sensor unit 106, MCU 114, actuator unit 101, and optionally transmitter unit 112. Fitting the protective housing unto the leg may be done by means of the attachment band. The attachment band is preferably of a design which will offer the user maximum comfort and which may be easily attached and removed.

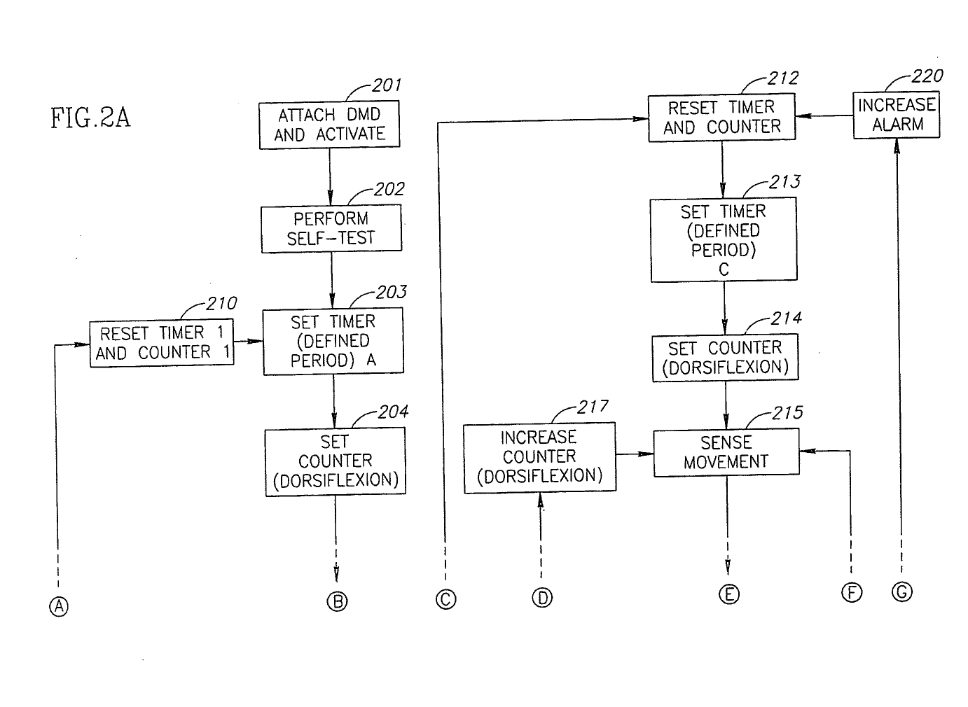

Reference is made to FIGS. 2a and 2b which schematically show a flow chart, divided in two portions, illustrating the method of operation of the DMD of FIG. 1 in accordance with an embodiment of the invention. The DMD is attached to the leg and activated 201. The MCU runs a self-test to check all functions and proper operation of the DMD 202. The timer, adapted to count down from a defined period A, is started 203. A counter adapted to count the number of dorsiflexion movements is started 204. The MCU starts to read data from the sensor unit 205. The MCU translates the data from the sensor unit and checks if a movement has been detected 206. If movement is detected the MCU increases the counter 207 and reads the sensor unit again (back to step 205). If movement is not detected the MCU checks if the timer has finished counting down 208. If timer countdown is not finished, the MCU returns to read data from the sensor unit (back to step 205). If timer countdown is finished, the MCU checks if the counter reached a pre-defined value B 209. If the counter reached the pre-defined value B, the MCU resets the timer and the counter 210 and returns to step 203. If the counter has not reached the pre-defined value B, the MCU triggers the actuator 211. The MCU resets the timer and the counter 212. The timer, adapted to count down from a defined period C, is initiated 213. The counter adapted to count the number of dorsiflexion movements is started 214. The MCU starts to read data from the sensor unit 215. The MCU translates the data from the sensor unit and checks if a movement has been detected 216. If a movement has been detected the MCU increases the counter 217 and reads the sensor unit again (back to step 215). If movement is not detected, the MCU checks if the timer has finished counting down 218. If the timer countdown is not finished, the MCU returns to read data from the sensor unit (back to step 215). If the timer countdown is finished, the MCU checks if the counter reached the pre-defined value B 219. If the counter has not reached the pre-defined value B the MCU signals the actuator to increase the intensity of the stimulating action 220 and resets the timer and the counter (back to step 212). If the counter reached the value B, the MCU stops the actuator 221 and resets the second timer and the counter (back to step 210).

In accordance with an embodiment of the invention the alarm is continuously activated during steps 11 through 21, its intensity augmented every C period of time. The parameters A, B, and C may be redefined for different users and/or for different scenarios. For example, for one user and/or scenario the parameters may have values A=20 minutes, B=5 minutes and C=30 sec, while for another user and/or scenario the parameters may have values A=60 minutes, B=15 minutes, and C=2 minutes.

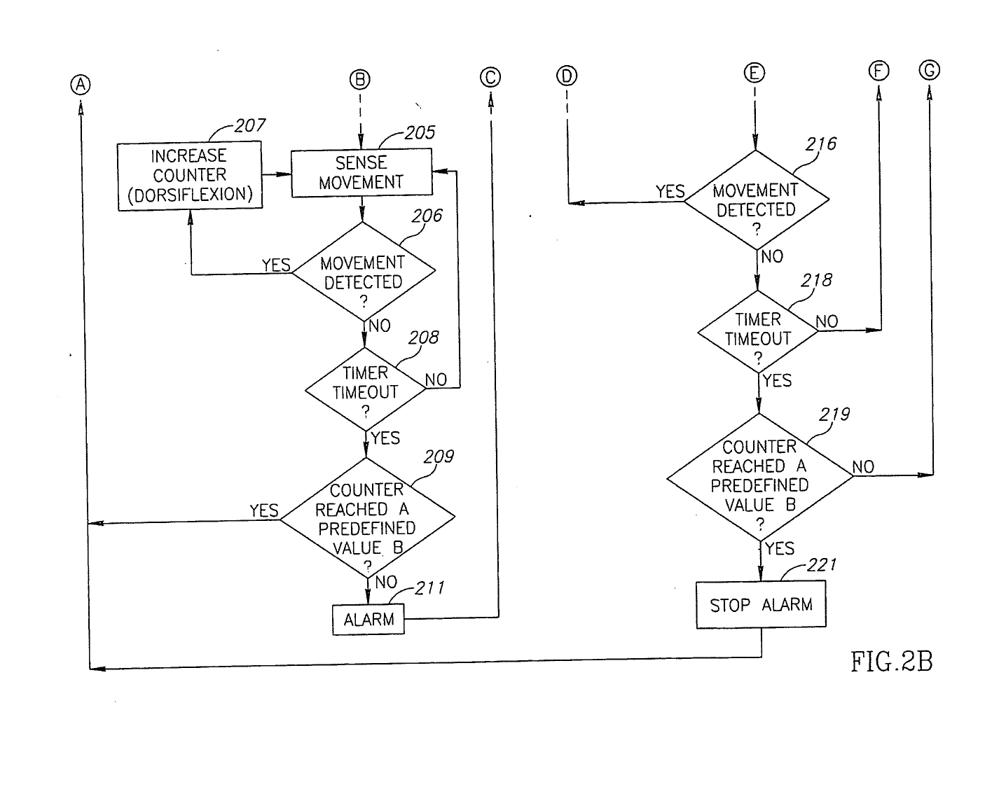

Reference is made to FIG. 3 which schematically shows a DMD 300 in an exemplary attachment configuration for sensing Anterior Tibial muscle contraction in a leg in response to dorsiflexion, in accordance with embodiment of the invention. Referring to an x-y axis 303 dorsiflexion is represented by the action of flexing a foot lying in a plane parallel to the x-axis in the direction of the y-axis.

DMD 300 comprises a protective case 301 and an attachment band 302, protective case 301 comprising a power unit (not shown), a sensor unit (not shown), a main control unit (MCU) (not shown), an actuator unit (not shown), and optionally a transmitter unit (not shown). Protective case 301 and attachment band 302 may be the same or substantially similar to the protective case and attachment band of DMD 100 shown in FIG. 1. The DMD, sensor unit, MCU, actuator unit, and transmitter may be the same or substantially similar to those shown in FIG. 1 at 100, 106, 114, 101, and 112, respectively. The power unit may be the same or substantially similar to that comprised in DMD 100 shown in FIG. 1.

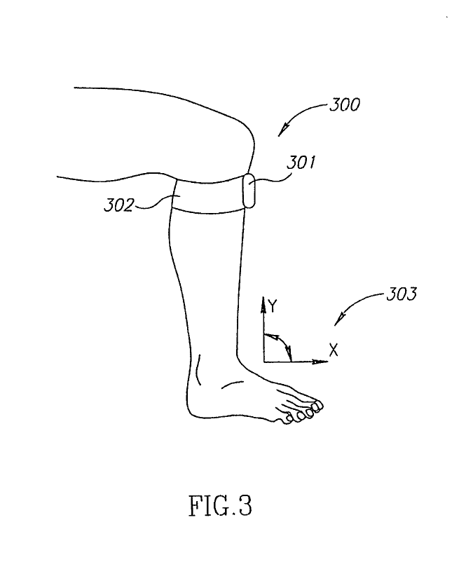

Reference is made to FIG. 4 which schematically shows a DMD 400 in an exemplary attachment configuration for sensing Achilles tendon and/or Anterior Tibial muscle tendon movement in the leg in response to dorsiflexion, in accordance with another embodiment of the invention. Referring to an x-y axis 403 dorsiflexion is represented by the action of flexing a foot lying in a plane parallel to the x-axis in the direction of the y-axis.

DMD 400 comprises a protective case 401 and an attachment band 402, protective case 401 comprising a power unit (not shown), a sensor unit (not shown), a main control unit (MCU) (not shown), an actuator unit (not shown), and optionally a transmitter unit (not shown). Protective case 401 and attachment band 402 may be the same or substantially similar to the protective case and attachment band of DMD 100 shown in FIG. 1. The DMD, sensor unit, MCU, actuator unit, and transmitter may be the same or substantially similar to those shown in FIG. 1 at 100, 106, 114, 101, and 112, respectively. The power unit may be the same or substantially similar to that in DMD 100 shown in FIG. 1.

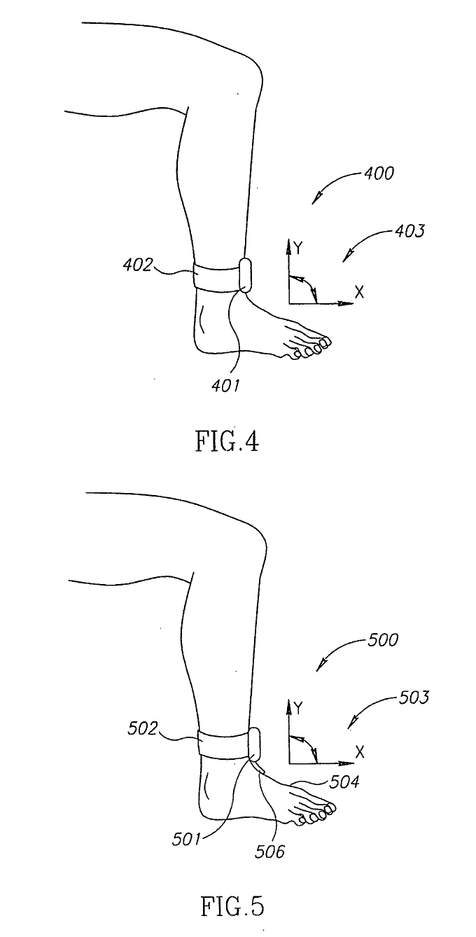

Reference is made to FIG. 5 which schematically shows a DMD 500 in an exemplary attachment configuration for sensing dorsiflexion directly from the dorsal side 504 of the foot, in accordance with another embodiment of the invention. Referring to an x-y axis 503 dorsiflexion is represented by the action of flexing a foot lying in a plane parallel to the x-axis in the direction of the y-axis.

DMD 500 comprises a protective case 501 and an attachment band 502, protective case 501 comprising a power unit (not shown), a sensor unit (not shown), a main control unit (MCU) (not shown), an actuator unit (not shown), and optionally a transmitter unit (not shown). The sensor unit includes a switch 506 which may be depressed or toggled by dorsal side 504 during dorsiflexion.

Protective case 501 and attachment band 502 may be the same or substantially similar to the protective case and attachment band of DMD 100 shown in FIG. 1. The DMD, sensor unit, MCU, actuator unit, and transmitter may be the same or substantially similar to those shown in FIG. 1 at 100, 106, 114, 101, and 112, respectively. The power unit may be the same or substantially similar to that in DMD 100 shown in FIG. 1.

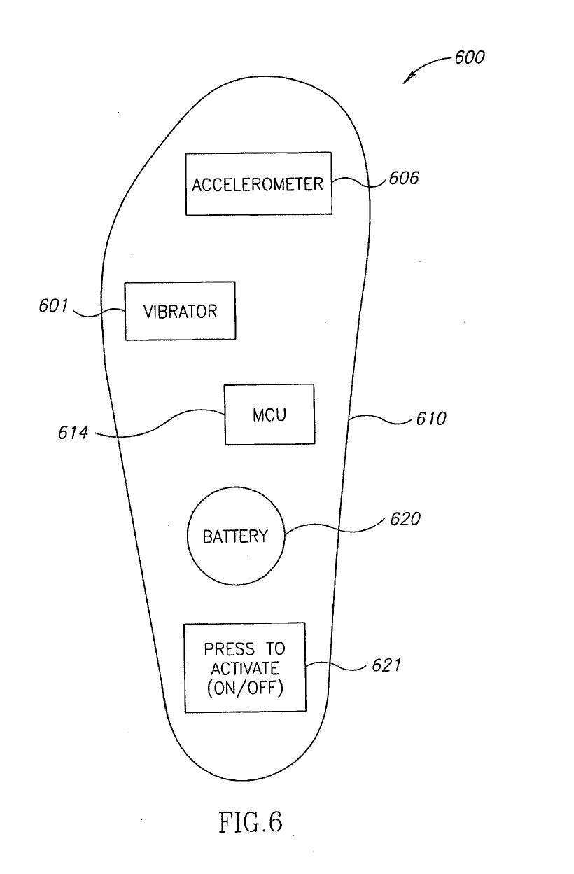

Reference is made to FIG. 6 which schematically shows a DMD 600 comprised in an exemplary insole 610 of a shoe for sensing dorsiflexion, in accordance with some embodiments of the invention. DMD 600 comprises a sensor unit 606, an actuator unit 601, an MCU 614, a power supply unit 620, and a switch 621. DMD 600, sensor unit 606, actuator unit 601, MCU 614, and power supply 620, are the same or substantially similar to that shown in FIG. 1 at 100, 106, 101, 114, and 120. Switch 621 is adapted to activate and deactivate the power in the DMD.

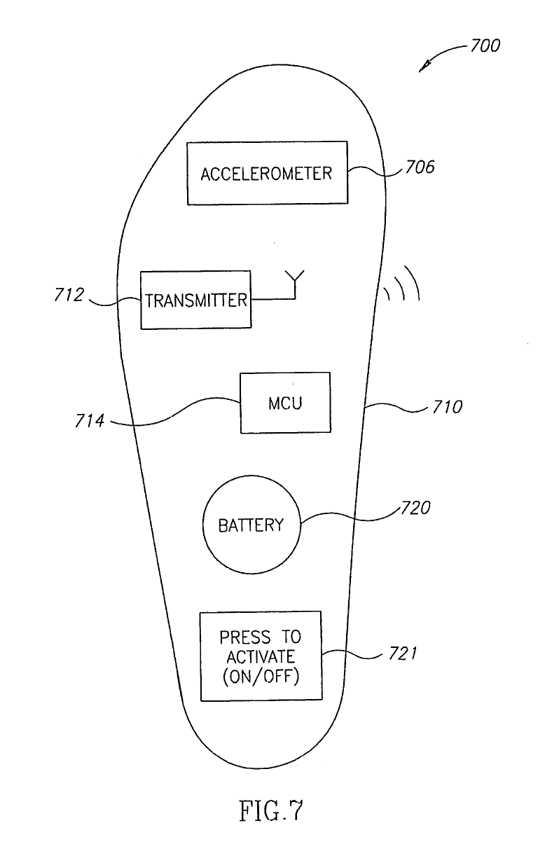

Reference is made to FIG. 7 which schematically shows a DMD 700 including an RF transmitter, comprised in an exemplary insole 710 of a shoe for sensing dorsiflexion, in accordance with some embodiments of the invention. DMD 700 comprises a sensor unit 706, a transmitter unit 712, an MCU 714, a power supply unit 720, and a switch 721. DMD 700, sensor unit 706, transmitter unit 712, MCU 714, and power supply 720, are the same or substantially similar to that shown in FIG. 1 at 100, 106, 712, 114, and 120. Switch 721 is adapted to activate and deactivate the power in the DMD.

In some embodiments of the invention the DMD is comprised in a shoe. Optionally, in some embodiments of the invention, the DMD is comprised in a sock.

In the description and claims of embodiments of the present invention, each of the words “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art. The scope of the invention is limited only by the claims.

What we claim is:

1. A device adapted for mitigating the formation of a blood clot, the device comprising:
a sensor adapted to identify muscle movement associated with blood circulation in a vein and to produce a signal indicating the movement; and
a controller adapted to monitor the sensor signal and to activate an actuator upon said controller determining that less than a defined number of movements have been sensed over a defined period of time, wherein the actuator is adapted to produce a stimulating action of a given set of parameters.

2. The device according to claim 1, wherein the muscle movement comprises dorsiflexion.

3. The device according to claim 1, wherein the number of muscle movements, the period of time or both are dynamically re-definable.

4. The device according to claim 1, wherein the number of muscle movements, the period of time or both are defined according to medical conditions of the user.

5. The device according to claim 1, wherein said controller is adapted to reset a counter upon said controller determining that a defined number of movements have been sensed over a defined period of time.

6. The device according to claim 1, wherein the controller is adapted to stop the activation of the actuator upon determining that at least defined number of movements have been sensed over a defined period of time.

7. The device according to claim 1, wherein the stimulating action comprises motion, vibration, rotation, alarm, electric pulse production or any combination thereof.

8. The device according to claim 1, wherein the given set of parameters comprises frequency, intensity, time or any combination thereof.

9. The device according to claim 1, wherein any one of the parameters is variable.

10. The device according to claim 1, wherein the controller is adapted to vary at least one parameter after a certain period of time has passed from the initiation of stimulating action.

11. The device according to claim 1, wherein said actuator is further adapted to be affixed near the sensed muscle and to stimulate a user when actuated.

11. The device according to claim 1, wherein said actuator comprises a mechanical actuator, an electric actuator, an aural actuator, visual actuator or any combination thereof.

12. The device according to claim 12, wherein the electric actuator is adapted to stimulate and trigger the muscle of a user.

13. The device according to claim 1, wherein said sensor comprises a mechanical sensing unit, a piezo-electric sensing unit, a strain gage sensing unit, an Infrared (IR) sensing unit, an acceleration sensing unit or any combination thereof.

14. The device according to claim 1, wherein said sensor is located in or in proximity to the dorsal side of the foot.

15. The device according to claim 1, wherein said sensor is adapted to sense the muscle through sensing motion of a tendon.

16. The device according to claim 1, wherein said sensor is adapted to sense the muscle through sensing a contraction or stretching of the muscle.

17. The device according to claim 1, further comprising a fastener adapted to attach said device to a users limb.

18. The device according to claim 1, further comprises a transmitter adapted to trigger an action of a device for stimulating blood flow in a foot.

19. The device according to claim 19, wherein said transmitter comprises a wireless transmitter.

20. The device according to claim 20, wherein said wireless transmitter comprises a radio frequency (RF) transmitter.

21. A shoe comprising the device according to claim 1.

22. A sock comprising the device according to claim 1.

23. An insole comprising the device according to claim 1.

24. A method of mitigating the formation of a blood clot, the method comprising:
sensing muscle movements associated with blood circulation in a vein; and
actuating a stimulating action of a given set of parameters upon determining that less than a defined number of movements have been sensed over a defined period of time.

25. The method according to claim 24, wherein the muscle movement comprises dorsiflexion.

26. The method according to claim 24, wherein the defined number of muscle movements is at least one and the period of time is above 1 minute.

27. The method according to claim 24, wherein the defined number of muscle movements, the period of time or both are dynamically re-definable.

28. The method according to claim 24, wherein the number of muscle movements, the period of time or both are defined according to medical and/or environmental conditions related to the user.

29. The method according to claim 24, further comprising resetting a counter upon determining that a defined number of movements have been sensed over a period of time.

30. The method according to claim 24, further comprising stopping the actuation upon determining that at least defined number of movements have been sensed over a period of time.

31. The method according to claim 24, wherein the stimulating action comprises vibration production, sound production, light production, electric pulse production or any combination thereof.

32. The method according to claim 24, wherein the given set of parameters comprises frequency, intensity, time or any combination thereof.

33. The method according to claim 24, wherein any one of the parameters is variable.

34. The method according to claim 24, further comprising varying at least one parameter after a certain period of time has passed from the initiation of the stimulating action

35. The method according to claim 24, further comprising stimulating blood flow in a foot.

36. A system adapted to increase blood flow comprising the device according to claim 1.
